(19) United States
(12) Patent Application Publication
(10) Pub. No.: US 2019/0290439 A1
(43) Pub. Date: Sep. 26, 2019

(54) Multi-Layered Osteochondral Construct and Subchondral Bone Analog Thereof

(71) Applicant: CLEMSON UNIVERSITY, Clemson, SC (US)

(72) Inventors: ALAN M. MARIONNEAUX, Clemson, SC (US); Jeremy J. Mercuri, Clemson, SC (US); Joshua D. Walters, Clemson, SC (US)

(21) Appl. No.: 16/292,386

(22) Filed: Mar. 5, 2019

Related U.S. Application Data
(60) Provisional application No. 62/638,530, filed on Mar. 5, 2018.

(51) Int. Cl. A61F 2/30; A61L 27/24; A61L 27/38

(52) U.S. Cl. CPC A61F 2/30756; A61L 27/24; A61L 27/3817; A61L 27/56; A61F 2002/30766; A61F 2002/30762; A61F 2002/30764

(57) ABSTRACT

Subchondral bone analog materials and osteochondral constructs that incorporate the subchondral bone analogs are described. The subchondral bone analog materials include a biodegradable matrix, calcium phosphate particles (e.g., hydroxy apatite) and bioactive glass particles. The materials can exhibit sufficient mechanical strength and biochemical properties such that the materials can support boney integration and healing. Osteochondral constructs can include a first layer of the subchondral bone analog material, a second layer of a calcified cartilage analog material, and a third layer of a cartilage analog material.

### CROSS REFERENCE TO RELATED APPLICATION

This application claims filing benefit of U.S. Provisional Patent Application Ser. No. 62/638,530, entitled “Tailoring the Subchondral Bone Phase of a Multi-Layered Osteochondral Implant to Support Bone Healing and a Cartilage Analog,” having a filing date of Mar. 5, 2018, which is incorporated herein by reference for all purposes.

### BACKGROUND

Focal chondral and osteochondral defects often result from joint wear and tear, traumatic injury, or metabolic disorders and create pain and disability for a significant portion of people, including working-aged adults. The quality of life for millions of individuals worldwide is detrimentally affected by focal chondral or osteochondral defects. For instance, between 2004 and 2011, approximately 200,000,000 incidences of focal chondral or osteochondral defects occurred in adults in the United States, with 2,000,000 incidences requiring surgical intervention. If left untreated, these defects result in persistent joint pain that limits the ability to perform daily, work, and recreational activities. Long-term, untreated defects can lead to further degenerative changes in the joint. This poses an especially challenging clinical problem because focal chondral defects are unable to heal intrinsically due to the absence of vasculature and the presence of a relatively immobile and quiescent resident cell population.

A variety of surgical approaches have been implemented in an attempt to repair damaged cartilage tissue. Osteoarticular transplantation (OAT) is one of the most commonly utilized surgical methods for chondral and osteochondral focal defect repair. A graft is generated through the removal of an osteochondral tissue plug from a non-weight-bearing region of the knee (autograft) or donor tissue (allograft) and is subsequently press-fit into the existing focal defect. While this technique uses healthy patient or donor bone and cartilage in an attempt to preserve joint physiology, it suffers from major drawbacks including: donor site morbidity, limited donor tissue availability, donor graft rejection, and bone/cartilage depth mismatch.

Off-the-shelf osteochondral constructs have also been developed and investigated as a potential method of overcoming limitations and improving patient outcomes. Unfortunately, these approaches have also failed to provide long-term repair attributed to a lack of cartilage integration, among other issues.

Previous development approaches for osteochondral constructs have been primarily focused on the overlaying cartilage layer with less effort devoted to the underlying subchondral bone at the repair site. However, insufficient osseous support is a recognized cause of clinical failure of osteochondral repair constructs. This lack of support is attributed either to a lack of mechanical integrity of the boney layer at the repair site or a failure of the repair construct to integrate with adjacent host bone.

What are needed in the art is a synthetic biocompatible subchondral bone analog that can mimic subchondral bone with regard to mechanical characteristics (e.g., support) as well as exhibit integration with natural tissues. Osteochondral constructs that incorporate the subchondral bone analog in conjunction with other analogs in order to more closely mimic the structure of tissues at osteochondral defect sites would also be of great benefit in the art.

### SUMMARY

According to one embodiment, disclosed is a synthetic biocompatible subchondral bone analog material that includes a biocompatible polymeric matrix, particles comprising a calcium phosphate in the polymeric matrix, and particles comprising a bioactive glass in the polymeric matrix.

Also disclosed is an osteochondral construct that includes a first layer that includes a synthetic biocompatible subchondral bone analog, a second layer that includes a calcified cartilage analog, and a third layer that includes a cartilage analog.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 schematically illustrates one embodiment of a multi-layered osteochondral implant as described herein.

FIG. 2 schematically illustrates one embodiment for crosslinking and compacting an artificial cartilage analog as described herein.

FIG. 3 compares mass loss of different non-compacted subchondral bone (ScB) materials after 28 days of degradation. In all figures, unless stated otherwise, bars or star (*) indicate significant difference between groups (p<0.05).

FIG. 4 compares mass loss of different ScB materials after 14 days of degradation.

FIG. 5 compares pH effects of ScB material degradation on modified simulated body fluid (mSBF) bathing solutions for different non-compacted ScB materials degraded up to 28 days.

FIG. 6 compares pH effects of ScB material degradation on mSBF bathing solutions for different ScB materials degraded up to 14 days.

FIG. 7 includes at A) representative images of the progressive stages of micro CT analysis for different non-compacted ScB materials; at B) is a graph illustrating mineral phase volume of different non-compacted ScB materials over time during degradation, and at C) is a graph depicting the biphasic distribution of each non-compacted ScB material following degradation.

FIG. 8 includes at A) representative images of the progressive stages of micro CT analysis for different ScB materials; at B) is a graph illustrating implant mineral phase volume of different ScB materials over time during degradation, and at C) is a graph depicting the biphasic distribution of different ScB materials following degradation.

FIG. 9 illustrates the change in soluble ion concentrations in mSBF bathing solutions for different non-compacted ScB materials over a degradation period. Ions included Calcium at A; Phosphorous at B; Silica at C; and Magnesium ions at D.

FIG. 10 illustrates the change in soluble ion concentrations in mSBF bathing solutions for different ScB materials over a degradation period. Ions included Calcium at A; Phosphorous at B; Silica at C; and Magnesium ions at D.

FIG. 11 provides compressive modulus values for different non-compacted ScB materials tested after 0, 14, 21, and 28 days of degradation.

FIG. 12 provides compressive modulus values of different ScB materials following a degradation protocol. Star (*) indicates significant difference compared to a particular non-compacted material (p<0.05) while pound (#) indicates significant difference compared to a particular compacted material.

FIG. 13 provides compressive modulus values of an ScB material at various annealing times. Star (*) indicates significant difference compared to 0 minute anneal time (p<0.05).

FIG. 14 provides compressive modulus values for different ScB materials at different times throughout a degradation protocol.

FIG. 15 illustrates fluorescent intensity of AB supplemented media after incubation with hMSC-BM seeded ScB material. An increase in RFU correlates with an increase in cellular proliferation and metabolism. Star (*) indicates significant difference from the Day 0 group (p<0.05).

FIG. 16 presents cell viability measures for multi-layered osteochondral plugs in an indirect cytotoxicity study.

FIG. 17 presents complex modulus values for multi-layer composites including a crosslinked cartilage analog layer over a sweep of frequencies.

FIG. 18 presents dynamic moduli values for multi-layer composites.

FIG. 19 presents interfacial shear mechanical properties of multi-layer composites.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

The present disclosure is directed to subchondral bone analog materials and osteochondral constructs that incorporate the subchondral bone analogs. Beneficially, the subchondral bone analog materials can exhibit sufficient mechanical strength and biochemical properties such that the materials can support boney integration and healing. Disclosed subchondral bone analog materials exhibit biochemical, biophysical, and bioactive characteristics that can support integration with natural tissues and promote bone healing in an implant area.

For instance, the subchondral bone analog materials can degrade and support integration of new healthy tissue without causing unintended negative consequences in the implant area. By way of example, the materials can degrade slowly, without excessive mass loss prior to suitable healing and integration. For instance, disclosed materials can exhibit a mass loss of about 10% or less, about 6% or less, or about 5% or less in some embodiments following degradation in a simulated body fluid for 28 days. In some embodiments, the subchondral bone analog materials can exhibit a mass loss of about 5% or less, about 4% or less, about 3% or less, about 2% or less, or about 1% or less following degradation in a simulated body fluid for 14 days.

Upon degradation of a subchondral bone analog material, the degradation products can have little or no effect on local pH and as such, can avoid triggering of an immune response to the implant materials. For instance, following degradation in a simulated body fluid for a period of 28 days, the simulated body fluid can retain a pH of 7.7 or less, or 7.6 or less, for instance between about 7.5 and about 7.6 in some embodiments.

In addition, the subchondral bone analog materials can exhibit measurable bioactivity in vitro, and as such can encourage integration to natural surrounding tissues. Moreover, the materials exhibit excellent mechanical characteristics so as to provide desirable osseous support to an implant. For instance, disclosed materials can have a compressive modulus of about 5 MPa or greater, about 10 MPa or greater, about 20 MPa or greater, about 50 MPa or greater, or about 150 MPa or greater in some embodiments, for instance from about 50 MPa to about 150 MPa in some embodiments.

When combined with other materials to form a multi-component osteochondral construct, the subchondral bone analog materials can securely attach to other materials of the construct. The other components of the construct can closely mimic the characteristics of other tissues in an osteochondral defect, e.g., cartilage, calcified cartilage. For instance, in one embodiment, an osteochondral construct can include a subchondral bone analog combined with an overlaying cartilage analog, and the different materials can be adhered/attached to one another so as to provide a strong, unitary, multilayer construct suitable for implant.

Osteochondral constructs that incorporate the subchondral bone analog materials can also exhibit excellent mechanical and biochemical characteristics. For instance, a multi-layer construct that includes a subchondral bone analog as one layer in conjunction with other analog layers can exhibit an excellent attachment strength across the unitary construct. By way of example, a multi-layer construct can exhibit an ultimate shear stress of about 500 kPa or greater, for instance from about 500 kPa to about 1000 kPa and can exhibit a shear stiffness of about 400 N/mm or greater, for instance from about 400 N/mm to about 700 N/mm.

A multi-layer osteochondral construct can also exhibit excellent compressive strengths. For instance, an overlaying cartilage analog of a multi-layer construct can exhibit a complex modulus of a about 50 kPa or greater, about 100 kPa or greater, or about 200 kPa or greater, for instance from about 100 kPa to about 300 kPa at a frequency of from about 0.1 Hz to about 50 Hz.

The osteochondral bone analog material includes a polymeric matrix that incorporates a biodegradable polymer. The matrix can generally include any biodegradable polymer as is commonly used, many of which have been thoroughly investigated for the use in biological implant applications. Representative biodegradable polymers can include, without limitation, homopolymers or copolymers of poly(lactic acid) (PLA) or poly(glycolic acid) (PGA) (e.g., poly(lactic-co-glycolic acid) (PLGA)), poly(ortho esters), polyanhydrides, polycaprolactones (PCL), polysulfones, polyolefins, polyvinyl alcohol (PVA), polyalkenoics, polyacrylic acids (PAA), polyesters, as well as combinations of two or more thereof. Polymers comprising poly(lactides), copolymers of lactides and glycolides, blends thereof, or mixtures thereof can be utilized in one embodiment. Such polymers can be formed from monomers of a single isomeric type or a mixture of isomers.

A polymeric matrix can include natural polymers such as proteoglycans, glycosaminoglycans, polysaccharides, proteins, and the like, as well as synthetic polymers. A non-limiting list of polymeric materials derived from natural materials that can be incorporated in a polymeric matrix can include, without limitation, dextran, hyaluronic acid, chitin, heparin, collagen, elastin, keratin, albumin, alginates such as sodium alginate or crosslinked alginate gum, polycaprolactone, polyanhydride, pectin, and polysaccharides, as well as combinations of two or more polymers, optionally in combination with one or more synthetic polymers, for instance in a polymeric blend, copolymerized or crosslinked with one another, or any combination thereof.

A non-biodegradable, biocompatible polymer can also be incorporated in a polymeric matrix in some embodiments. Examples of which can include, without limitation, biocompatible polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, as well as blends and copolymers thereof.

In general, the polymeric matrix can be highly hydrated while maintaining structural stability. Suitable matrices can include noncrosslinked and crosslinked matrices as well as matrices that incorporate crosslinked portions in conjunction with noncrosslinked (e.g., blended) portions. In general, a polymeric matrix can include hydrolyzable portions, such that the matrix can be degradable when utilized in an aqueous environment, e.g., in vivo.

A subchondral bone analog material can generally include a polymeric matrix in an amount of from about 15 wt. % to about 50 wt. % of the analog material, for instance from about 20 wt. % to about 45 wt. %, or from about 35 wt. % to about 45 wt. % in some embodiments.

The polymeric matrix of the subchondral bone analog material can include particulate additives that can improve the biochemical and physical characteristics of the material. The particulate additives can include calcium phosphate particles and bioactive glass particles.

Various calcium phosphates are encompassed and can include, without limitation, tricalcium phosphates, (e.g., 13-tricalcium phosphate and a-tricalcium phosphate), as well as apatites such as hydroxyapatite. An exemplary calcium phosphate product is hydroxyapatite available from CaP Biomaterials, LLC of East Troy, Wis.

In general, the calcium phosphate particles can be porous and can include micro-, meso-, or macro-porosity or any combination thereof. Macro-porosity is generally characterized by pore diameters greater than about 100 μm, for instance, from about 100 μm to about 2000 μm. Meso-porosity is generally characterized by pore diameters between about 10 μm and about 100 μm, and micro-porosity is generally characterized by pore diameters of about 10 μm or less. In general, the porosity of the calcium phosphate particles can be interconnected.

The relative proportions of different classifications of porosity in the calcium phosphate particles is not generally critical. However, in some embodiments, the overall porosity of the calcium phosphate particles can be high, for instance about 30% or more, about 50% or more, about 60% or more, about 70% or more, about 75% or more, about 80% or more, or about 90% or more. Pore volume can be measured in one embodiment by Helium Pycnometry. As is known in the art, this procedure determines the density and true volume of a sample by measuring the pressure change of helium in a calibrated volume. Porosity and pore size distribution may also be measured by mercury intrusion porosimetry as is known.

The calcium phosphate particulate of a subchondral bone analog material can be relatively large. For instance, the subchondral bone analog material can include particles in a size range of from about 1000 μm to about 2000 μm. For instance, about 90 wt. % or more of the calcium phosphate particles can be in a size range of from about 1000 μm to about 2000 μm, though the particulate can incorporate an amount of calcium phosphate particles that fall outside of this size range. Unless otherwise specified, particle size as used herein refers to the sieve size used to partition the glass particles. For instance, a particulate in which 90 wt. % or more of the particles are larger than 1000 microns would encompass a particulate in which at least 90% of the particles will be retained on a screen having mesh openings of 1000 microns.

In general, a subchondral bone analog material can include the calcium phosphate particles in an amount of from about 25 wt. % to about 50 wt. %. For instance from about 30 wt. % to about 45 wt. % or from about 35 wt. % to about 40 wt. % in some embodiments.

In conjunction with the calcium phosphate particles, a subchondral bone analog material can also include particles that incorporate a bioactive glass. As utilized herein, the term “Bioactive glass” generally refers to alkali-containing ceramic, glass, glass-ceramic, or crystalline material that reacts as it comes in contact with physiologic fluids including, but not limited to, blood and serum. Examples of bioactive glasses include those described in U.S. Pat. No. 5,914,356, to Erbe, and U.S. Pat. No. 6,709,744 to Murphy, et al., incorporated herein by reference. Suitable bioactive materials also include 45S5 glass and glass-ceramic, 58S5 glass, S53P4 glass, 13-93 glass, apatite-wollastonite containing glass and glass-ceramic. Examples of bioactive glass as may be included in a subchondral bone analog material include those available from the Mo-Sci Corporation. For instance, a bioactive glass particulate can include SiO₂ in an amount of from about 40 wt. % to about 55 wt. %, CaO in an amount of from about 20 wt. % to about 25 wt. %, Na₂O in an amount of from about 5 wt. % to about 25 wt. %, P₂O₅ in an amount of from about 1 wt. % to about 10 wt. %, and optionally including from about 5 wt. % to about 15 wt. % K₂O and/or up to about 10 wt. % MgO.

While not wishing to be bound by theory, it is believed that the bioactive glass component of the subchondral bone analog material may be stimulatory to osteoblasts.

The particle size of the bioactive glass particulate is not particularly limited, For instance, in some embodiments, bioactive glass particles of a subchondral bone analog material can include particles between about 20 μm and about 200 μm, e.g., about 90 wt. % or more of the particulate will have a particle size between about 20 μm and about 200 μm. The bioactive glass particulate may include particles of about 150 μm or less, for instance about 100 μm or less, in some embodiments. The bioactive glass particles may be solid or may be porous, though in general, the bioactive glass can be nonporous.

In general, a subchondral bone analog material can include a bioactive glass particulate in an amount of from about 20 wt. % to about 40 wt. %, for instance from about 20 wt. % to about 30 wt. % in some embodiments.

To form a subchondral bone analog material, a biocompatible polymeric composition can be combined with the particulate additives. For instance, a solution comprising the polymers of the matrix can be combined with the calcium phosphate particles and the bioactive glass particles by blending to form a substantially homogenous mixture. As used in this context, “substantially homogenous” means that the ratio of components within the mixture is the same throughout.

In one method, a solution of the polymeric composition can be formed, and the particulate additive can then be combined with the solution. A polymeric solution can incorporate a solvent for the polymer that, in one embodiment can exhibit high water solubility, such as acetic acid, acetone, N,N-dimethylacetamide (DMAC), N-methylpyrrolidone (NMP), and tetrahydrofuran (THF). Following, the particles can then be combined with the solution. In general, the ratio of polymer to solvent can be from about 2:3 to about 1:20, though variations are encompassed herein.

The mixture can then be cast into a container and the solvent can be removed, e.g., by drying at room temperature and/or by heating. In some embodiments, the container can define the desired shape of the analog. In other embodiments an analog can be shaped, e.g., cut, from a large section following formation.

In some embodiments, a subchondral bone analog material can be further processed, for instance by compaction and/or annealing, which can modify characteristics of an implant. For instance, in one embodiment, prior to removal of all of the solvent from a mixture, the mixture including the polymer and the particulate additives can be subjected to compression. For instance, the mixture can be placed under a load of about 10 lb-f or less, e.g., from about 2 lb-f to about 5 lb-f, for a period of time of from about 2 minutes to about 10 minutes, which can serve to compact the matrix prior to final drying of the material.

In some embodiments, a subchondral bone analog material can be subjected to a heat treatment (i.e., annealed) following removal of the solvent and optionally, also following compaction and/or shaping of the material. For instance, a subchondral bone analog material can be held in an oven at a temperature of from about 100° C. to about 125° C., e.g., from about 110° C. to about 120° C. for a period of from about 30 minutes to about 5 hours, e.g., from about 1 hour to about 4 hours.

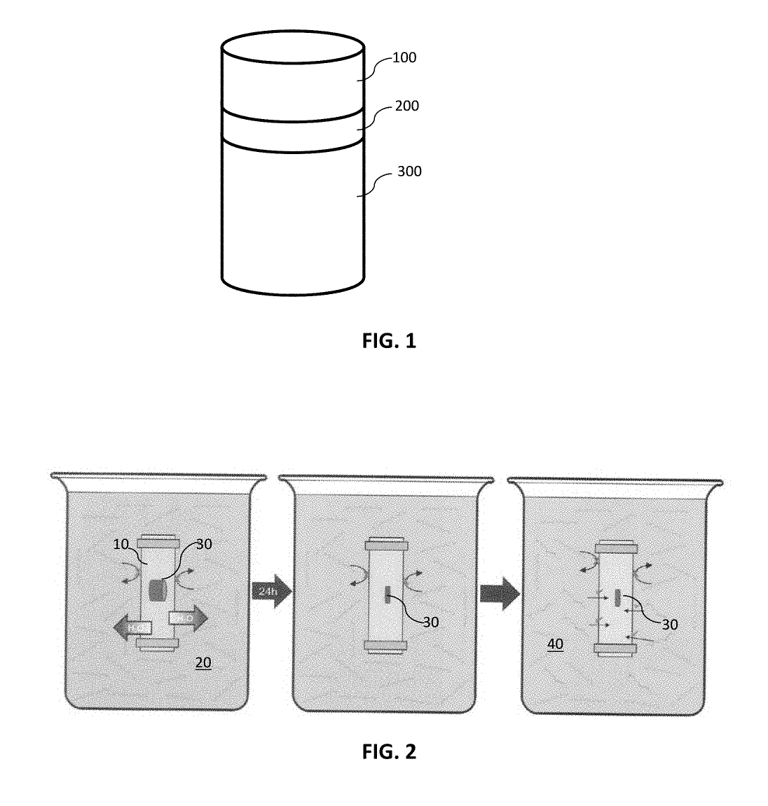

The subchondral bone analog material can be combined with one or more additional materials to form a multi-layered osteochondral implant. For instance, and with reference to FIG. 1, one embodiment of an osteochondral implant is illustrated that includes a first layer 100 that can include a cartilage analog, a second layer 200 that can include a calcified cartilage analog, and a third layer 300 that can include a subchondral bone analog.

An implant can be formed to any size and shape as is desirable, generally depending on the specific nature of the area in which the implant is intended to be located. For instance, an implant can be designed as an osteochondral defect implant, a scaffold for an osteochondral defect, a scaffold for bone tissue regeneration and reconstruction that promote vascularization and bone tissue ingrowth. The uses of the osteochondral implants are manifold. In one or more embodiments they may be useful for: bone void fillings or augmentation in zones requiring osteochondral bone; filling of bone defects after trauma, reconstruction, or correction in non-load or load-bearing indications; trauma and orthopedics; filling of voids caused by cysts or osteotomies, filling of defects arising from impacted fractures, refilling of cancellous bone-harvesting sites, arthrodesis and non-unions; spine surgery: posterolateral fusion, interbody fusion (as cage-filling material), vertebrectomies (as filling material of the vertebral implants), refilling of bone graft-harvesting sites, or cranio-maxillofacial surgery. As such, the size and shape of an implant can vary, and is not limited to any particular size or shape.

In this embodiment, a layer 200 adjacent to the subchondral bone analog layer 300 can include a calcified cartilage layer.

In natural osteochondral tissues, calcified cartilage is delineated from the overlaying cartilage via the tidemark and from the underlying subchondral bone via the cement line. It plays an important role mechanically because it transfers the load from the compliant cartilage tissue (E=1.9-15 MPa) to the stiff subchondral bone tissue (E=1.15-3.06 GPa). Additionally, the calcified cartilage is important biochemically. It is permeable to the transport of small molecules and mediates the biochemical interaction between the articular cartilage and subchondral bone. This helps stabilize the cartilage environment and maintain tissue homeostasis. The chondrocytes in this region express signs of hypertrophy and have a different phenotype than those in the non-calcified cartilage layer. Through incorporation of a region in an osteochondral implant that mimics the calcified cartilage region of natural tissue, integration of the surrounding tissues with the implant materials and successful rebuilding of a defect can be greatly improved.

A calcified cartilage analog material can include a polymeric matrix that incorporates a biodegradable polymer. Examples of polymers as may be encompassed in a polymeric matrix of a calcified cartilage analog can include any polymers or combinations thereof as previously described for a subchondral bone analog material. In one embodiment, the polymeric matrix of a calcified cartilage analog material can be the same as the polymeric matrix of the subchondral bone analog material of an osteochondral implant that incorporates the two materials, but this is not a requirement and in other implants, the two layers can include different polymer matrices that can differ as to one or more polymers included in the matrix, relative amounts of different polymers included in the matrix, size of polymers included in the matrix, amount of crosslinking between polymers of the matrix, and so forth.

A calcified cartilage analog material can also include calcium phosphate particles within the polymer matrix. The calcium phosphate particles can be the same calcium phosphate material as included in the calcium phosphate particulate of the subchondral bone analog material of the implant or can be different, as desired. For instance, the subchondral bone analog material can include hydroxy apatite and the calcified cartilage analog material can include a tricalcium phosphate. However, in some embodiments, both materials of a single osteochondral implant can include the same calcium phosphate material, e.g., hydroxy apatite.

The calcium phosphate particulate of a calcified cartilage analog material can be smaller than that of a subchondral bone analog material. For instance, a calcified cartilage analog material can include a large proportion, e.g., about 90 wt. % or more, about 95 wt. % or more or about 99 wt. % or more of the calcium phosphate particulate as particles of about 100 μm or less in size. For instance, the calcium phosphate particulate additive of a calcified cartilage analog material can be a calcium phosphate powder, e.g., a hydroxy apatite powder, in which all of the particles are about 100 μm or less in size.

A calcified cartilage analog material can include a relatively large portion of the calcium phosphate particulate in the material. For instance, a calcified cartilage analog material can include the calcium phosphate particulate in an amount of about 50 wt. % or more by weight of the material, for instance from about 50 wt. % to about 90 wt. %, from about 60 wt. % to about 90 wt. %, or from about 70 wt. % to about 80 wt. % in some embodiments,

A calcified cartilage analog material can be formed by combining a solution including the biocompatible polymeric composition with the particulate additive. For instance, a solution including the polymers of the matrix can be combined with the calcium phosphate particles by blending to form a substantially homogenous mixture.

For example, a solution of the polymeric composition can be formed, and the particulate additive can then be combined with the solution. A polymeric solution can incorporate a solvent for the polymer that, in one embodiment can exhibit high water solubility, such as acetic acid, acetone, N,N-dimethylacetamide (DMAC), N-methylpyrrolidone (NMP), and tetrahydrofuran (THF). Following, the particles can then be combined with the solution.

In forming an osteochondral implant a calcified cartilage analog material can be layered on a surface of a subchondral bone analog material following drying and any optional compression and/or annealing of the subchondral bone analog material. The amount of the calcified cartilage analog material to be applied to the surface of the subchondral bone analog material can vary, generally depending upon the intended application of the implant. In general, an implant can be designed so as to mimic the natural osteochondral tissue of a particular site, and as such the relative amounts of the various materials can be based upon the relative amounts of the natural materials present in a natural setting. By way of example, a calcified cartilage analog material can be layered on a surface of a subchondral bone analog material at a thickness of from about 50 microns to about 150 microns.

Referring again to FIG. 1, in conjunction with a layer 300 including a subchondral bone analog material and a layer 200, an osteochondral implant can also include a layer 100 that includes a cartilage analog material 100. It should be understood that presence of all three layers is not required in an osteochondral implant and the presence of one or more additional layers in conjunction with a subchondral bone analog material can depend upon the particular application of an implant.

In one embodiment, a cartilage analog material can be a decellularized natural tissue that exhibits the desirable biocompatible and biomechanical characteristics so as to mimic a natural cartilage material in an implant site. For instance, a cartilage analog material as described in U.S. Patent Application Publication No. 2018/0256784, which is incorporated herein for all purposes.

In one embodiment, a cartilage analog material can incorporate decellularized intervertebral disc tissue. The decellularized intervertebral disc tissue can be an allograft or xenograft intervertebral disc tissue that has been treated so as to be substantially free of cell nuclei. For instance, a cartilage analog material can be a processed natural tissue that has been processed so as to have a glycosaminoglycan content of about 200 micrograms (μg) or greater per milligram (mg) dry weight of the material and can include nucleic acids in an amount of about 50 nanograms (ng) or less per mg dry weight of the cartilage analog material. In one embodiment, the cartilage analog material can include nucleus pulposus tissue and/or articular cartilage (e.g., hyaline cartilage) that has been subjected to a decellularization process. The source tissue material for development of a cartilage analog material can be obtained from any xenogeneic or allogeneic source including, without limitation, porcine, bovine, human cadaver, etc.

By way of example, a natural tissue, e.g., a nucleus pulposus tissue or articular cartilage tissue, can be decellularized according to a chemical treatment process that includes contacting the tissue with a decellularization solution that includes one or more non-ionic surfactants and a protease inhibitor, optionally in conjunction with additional materials (e.g., antimicrobials, ionic surfactants, etc.). Examples of non-ionic surfactants can include, without limitation, an polyethylene oxide based surfactant or a polysorbate based surfactant such as Triton™ X-100 (t-octylphenoxypolyethoxyethanol), Tween® 20 (polysorbate 20), Tween® 80 (polysorbate 80), Igepal® CA630 (ethoxylated nonylphenol), etc. In general, the decellularization solution can include a non-ionic surfactant in an amount of about 0.5 v/v %, for instance from about 0.5 v/v % to about 2 v/v % in some embodiments.

Protease inhibitors can be included in a decellularization solution to prevent degradation of the extracellular matrix of the natural starting tissue. Suitable protease inhibitors can include, without limitation, N-ethylmaleimide (NEM), phenylmethylsulfonylfluoride (PMSF), ethylenediamine tetraacetic acid (EDTA), ethylene glycol-bis-(2-aminoethyl(ether)NNN′N′-tetraacetic acid, ammonium chloride, elevated pH, apoprotinin and leupeptin. In general, the decellularization solution can include a protease inhibitor in an amount of from about 0.1 w/v % to about 1 w/v %, for instance, from about 0.2 w/v % to about 0.6 w/v %. A decellularization solution can include additional components as are generally known in the art including, without limitation, one or more salts (e.g., KCl, NaCl), one or more organic or inorganic buffers, one or more antibiotics/antimycotics (e.g., penicillin, vancomycin, streptomycin, gentamycin, kanamycin, neomycin, sodium azide (NaN₃)) with or without anti-fungal agents (e.g., Amphotericin B, Nystain), etc.

A decellularization solution can be agitated while the tissue is held in contact with the solution. In addition, the decellularization solution containing the tissue can be subjected to ultrasonication. For instance, the solution can be periodically subjected to ultrasonication for a period of time, generally from about 5 minutes to about 60 minutes, at a power level of about 10 W to about 1000 W and at a frequency of from about 20 kHz to about 200 kHz. By way of example, a suitable ultrasonication condition may be from about 50 W to about 100 W at about 20 kHz to about 50 kHz for about 10 minutes.

Following contact and optional ultrasonciation, the tissue can be contacted with an enzyme solution, for instance to remove remaining DNA and RNA. A nuclease enzyme can generally be used to breakdown any remaining nucleic and ribonucleic acids. Enzymes can include nucleases such as endonucleases (e.g., DNAse, RNAse, Benzonase). An enzyme treatment can generally be carried out according to standard procedures as are known in the art.

Optionally, the cartilage analog material can be crosslinked. Any suitable crosslinking agent or method can be utilized. For example, collagen fixatives such a glutaraldehyde, carbodiimide, polyepoxides, etc. and/or elastin fixatives including polyphenolic compounds (tannic acid, pentagalloyl glucose, etc.) and the like can be utilized to cross-link the structural proteins of a cartilage analog material.

In one embodiment, a cartilage analog material can be compacted and optionally crosslinked according to a hyperosmotic pressure approach, which can also serve to compress and crosslink polymers of the cartilage analog material, which can improve characteristics of the material in some embodiments. On embodiment of a hyperosmotic pressure treatment is illustrated in FIG. 2. As can be seen a previously formed cartilage analog 30, e.g., a decellularized natural tissue formed as described previously can be held, for instance in a flexible container 10 that can optionally be porous to one or more components of a hyperosmotic solution 20 under agitation for a period of time, e.g., about 24 hours. A hyperosmotic solution can include as an osmotic component a material that can apply an osmotic pressure to the tissue without having any specific interactions with the tissue that could damage the cartilage analog material. For instance, in one embodiment a hyperosmotic solution can include a polymer such as polyethylene glycol or dextran, which can replace a significant portion of the water in/around the tissue and induce an osmotic pressure on the tissue. Based upon the particular characteristics of the system (e.g., size and amount of polymer, pH of the solution, biochemical composition of the cartilage analog, etc.), the hyperosmotic solution can produce a compressive osmotic pressure on the cartilage analog 30 of from about 0.01 MPa to about 5 MPa, which can compress the cartilage analog 30 as illustrated in the middle panel of FIG. 2.

In one embodiment, a cartilage analog can also be crosslinked according to a hyperosmotic crosslinking process. As indicated in FIG. 2, according to this embodiment, a crosslinking agent 40, a carbodiimide crosslinking agent, a succinimide crosslinking agent, glutaraldehyde, pentagalloyl glucose, etc., or combinations of crosslinking agents, can be added to a hyperosmotic solution 20 and under the osmotic pressure conditions, can contact the cartilage analog 30 to crosslink proteins of the cartilage analog, thereby adding further aiding stabilization of the cartilage analog 30.

A cartilage analog material can be securely adhered to other components of an osteochondral construct merely through application of force, which can compact the cartilage analog layer 100 with the other layers 200, 300 and form a unitary implant with the various layers strongly adhered to one another. By way of example, application of a force of from about 10 lb-f or less, e.g., from about 2 lb-f to about 5 lb-f, for a period of time of from about 10 seconds to about 5 minutes to securely bond the layers to one another.

Optionally, following attachment of the different layers to one another, a multilayer osteochondral construct can be further processed, for instance according to a heat treatment. For instance, an osteochondral construct can be held in an oven at a temperature of from about 100° C. to about 125° C., e.g., from about 110° C. to about 120° C. for a period of from about 30 minutes to about 5 hours, e.g., from about 1 hour to about 4 hours.

The subchondral bone analog materials and osteochondral constructs that incorporate the materials can exhibit mechanical, biophysical, and biochemical properties that may support bone healing. Utilization of disclosed materials and constructs may circumvent the limitations of other treatment strategies and improve clinical outcomes for a significant number of patients suffering from focal chondral and osteochondral defects.

The present disclosure may be better understood with reference to the Examples set forth below.

For all examples, GraphPad Prism 7 software was used for all statistical analyses. Results are represented as mean±standard error of the mean (SEM). Results were compared via two-way analysis of variance (ANOVA) followed by Tukey's post hoc analysis for multiple comparisons (pH, mass loss, morphological analysis, unconfined compression, bioactivity, and DMA). Interfacial shear testing was evaluated with a Student's t-test. Cytotoxicity data (live/dead and AB) was evaluated with a one-way ANOVA followed by a Dunnet's post hoc analysis for multiple comparisons against a control of cells cultured in standard cell culture media in parallel to experimental groups. Statistical significance was defined as (p<0.05) for all results. For all examples, formation materials included a 75:25 lactide to glycolide poly(D,L-lactide-co-glycolide) (PLGA) obtained from Lactel, Birmingham, Ala.; granulated hydroxyapatite (HAp) obtained from CaP Biomaterials LLC, East Troy, Wis.; and Bioactive Glass 13-93 powder (BG1393) (25-150 μm diameter) obtained from Mo-Sci Corporation, Rolla, Mo.

### EXAMPLE 1

Varying amounts of PLGA were dissolved in 15 mL of acetone and combined with 12 g of HAp (1000-1700 μm diameter) and 9 g of a BG1393 as shown in Table 1, below. The HAp and the BG1393 were thoroughly mixed into each of the PLGA solutions. The mixtures were cast into 50 mm diameter perfluoroalkoxy alkane (PFA) dishes and dried for 72 h at room temperature (RT) followed by 48 h at 60° C. Cylindrical implants were subsequently created using a 10 mm stainless steel punch. Implants were stored under desiccation until needed.

TABLE 1

| | PLGA1 | PLGA2 | PLGA3 |
|---|---|---|---|
| PLGA [g] | 15 | 10 | 5 |
| HAp [g] | 12 | 12 | 12 |
| BG1393 [g] | 9 | 9 | 9 |

To form compacted ScB implants (PLGA1C), following casting and prior to drying of a PLGA1 composition, mixtures were subjected to a defined compressive load of 5 lbs in order to compact the matrix. Cylindrical PLGA1C implants were then annealed in an oven at 115° C. for 30, 60, 120, or 240 minutes. PLGA1C implants that were annealed for 240 minutes were designated as PLGA1CA.

Modified simulated body fluid (mSBF) was prepared as described previously (Oyane et al., J. Biomed. Mater. Res. 65A (2003) 188-195) and adjusted to a pH of 7.50. All mSBF solutions were made on Day 0 of the respective degradation study in order to maximize solution stability.

All implants were weighed prior to being bathed in 35 mL of mSBF at 37° C. Bathing solution pH was recorded every two days for the study duration. At 0, 7, 14, and 28-day time points, implants (n=3/time point/formulation) were dried and weighed. Mass loss was calculated as:

$$\frac{Mass ( Day 0 ) - Mass ( time point )}{Mass ( Day 0 )} \times 100$$

Implants were stored under desiccation and their respective bathing solutions frozen at −80° C. for further analysis.

Implants were imaged using a SkyScan 1176 micro computed tomography (pCT) system (Bruker Corporation, Billerica, Mass.) at a resolution of 18 μm. To monitor the effect of degradation on biphasic morphology, non-compacted implants (n=3/formulation/time point) were imaged subsequent to degradation at 0, 7, 14, and 28 days, while compacted and compacted/annealed implants (n=3/formulation/time point) were imaged subsequent to degradation at 0, 7, and 14 days. Sample reconstruction was consistently performed over a defined cylindrical volume of interest (VOI) based around the central axis and cross-section of each implant for normalization purposes. Three-dimensional (3D) analysis was conducted for each VOI to quantify the volume of the implant occupied by the mineral phase and the distribution of the polymer phase throughout implant height (homogeneity).

The mSBF solutions bathing the ScB implants were removed from −80° C. storage, thawed to RT, and vortexed to ensure thorough mixing prior to analysis. Inductively Coupled Plasma Atomic Emission Spectroscopy (ICP-AES) was performed (PerkinElmer 3100 FL Atomic Absorption Spectrometer, PerkinElmer, Waltham, Mass.) to quantify calcium, phosphorous, silicon, and magnesium ion concentrations. Results are depicted as change in ion content relative to Day 0 mSBF controls.

The mechanical properties were evaluated using unconfined compression. Testing was conducted in a saline bath using an Instron 8872 servohydraulic mechanical test frame (Instron, Norwood, Mass.) equipped with a 25kN load cell. A 5N preload was applied to ensure implant contact. Implants were tested to failure or 65% strain (whichever occurred first) at a rate of 5 mm/min. Elastic modulus was calculated using a linear fit of the stress/strain graph (0-40% strain for non-compacted implants, 0-10% strain for all other implants).

ScB implants were sterilized with ethylene oxide (EO) using an Anprolene AN74i tabletop sterilizer (Andersen Products Inc., Haw River, N.C.) for 12 hours, followed by a 48-hour degassing period. Each implant was dynamically seeded with P3 human bone marrow derived mesenchymal stromal cells (hMSC-BM). The hMSC-BMs were suspended in cell culture medium (CCM) (Dulbecco's' Modified Eagle Medium [DMEM], 10% v/v fetal bovine serum [FBS], 1% v/v antibiotic and antimycotic [Ab/Am], and 1% v/v L-Glutamine [GLUT]) at a concentration of 1×10⁶ cells per 3 mL CCM. One sterilized ScB implant and 3 mL of cell suspension were placed in a sterile 50 mL conical tube and placed on an orbital shake plate in an aseptic laminar flow hood for 3 h at 100 rpm. Seeded implants were subsequently removed and individually placed into the wells of a non-tissue treated 12-well plate, submerged in CCM, and incubated under standard conditions (37° C. and 5% carbon dioxide buffer). Non-seeded implants were incubated in parallel with seeded implants for use as a negative control. The viability of hMSC-BM seeded implants was quantified at 0, 3, 7, and 14 days with an alamarBlue® assay (AB) (BioSource International, Camarillo, Calif.). Implants were incubated for 5 hours in CCM supplemented with 10% (v/v) AB. Fluorescence of AB was measured at an excitation wavelength of 545 nm and an emission wavelength of 590 nm using a fluorescence microplate reader (Synergy HT Multi-Mode Microplate Reader, Bio-Tek, Winooski, Vt.). Non-seeded implant values were used as a negative control and their fluorescent intensity was subtracted from experimental values at each time point. Cell viability was further verified at 0, 3, 7, and 14 days with live/dead fluorescent staining. After removing AB supplemented CCM, implants were rinsed 3× with sterile PBS then submerged in a solution of 2 μM calcein acetotoxymethyl, 4 μM ethidium homodimer-1, and 1 μL 4,6-Diamidino-2-phenylindole (DAPI) in PBS for 45 minutes at room temperature. Implants were rinsed and submerged in PBS and imaged with a fluorescent microscope (EVOS FL, Advanced Microscopy Group, Bothell, Wash.).

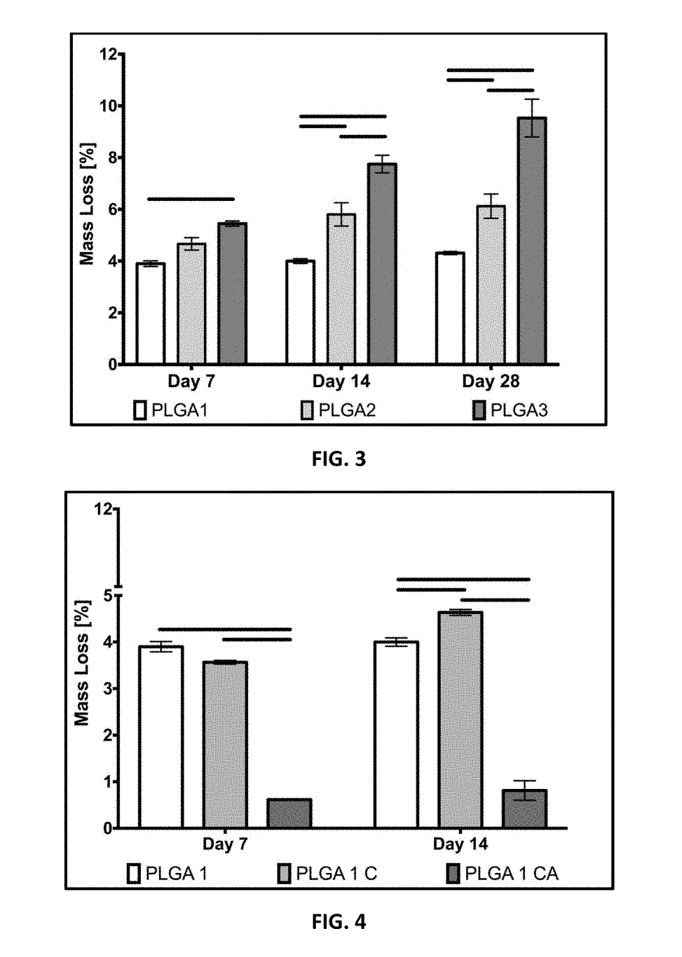

ScB mass loss following incubation in mSBF was evaluated to determine degradation kinetics. Comparing across the non-compacted implant groups, mass loss (FIG. 3) for PLGA1 (3.9±0.11%) was lower than PLGA2 (4.7±0.24%, p=0.3167) and significantly lower than PLGA3 (5.5±0.10%, p=0.0181) at Day 7. At Day 28, mass loss for PLGA1 (4.3±0.06%) was significantly lower than both PLGA2 (6.1±0.47%, p=0.0060) and PLGA3 (9.5±0.73%, p<0.0001). Comparing across time points, mass loss from Day 0 to Day 28 increased significantly for PLGA2 (p=0.0261) and PLGA3 (p<0.0001). Particulate was readily apparent in the bottom of the test tubes containing the mSBF and non-compacted implants.

Mass loss for compacted implants (PLGA1C) (3.6±0.04%) was marginally lower compared to PLGA1 (3.9±0.11%) at Day 7 (p=0.1136) (FIG. 4). Mass loss for PLGA1C (4.6±0.06%) was significantly greater compared to PLGA1 (4.0±0.09%) at Day 14 (p=0.0034). Comparing across time points, mass loss from Day 0 to Day 14 was relatively constant for PLGA1C. Particulate was readily apparent in the bottom of the test tubes containing the mSBF and compacted implants.

Mass loss for compacted and annealed implants (PLGA1CA) (0.6±0.01%) was significantly lower compared to PLGA1C (3.6±0.04%) at Day 7 (p<0.0001) (FIG. 4). Mass loss for PLGA1CA (0.8±0.21%) was significantly lower compared to PLGA1C (4.6±0.06%) at Day 14 (p<0.0001). Comparing across time points, mass loss from Day 0 to Day 14 significantly increased for PLGA1CA (p<0.0001). Particulate was not noted in the tubes containing mSBF and the compacted and annealed implants.

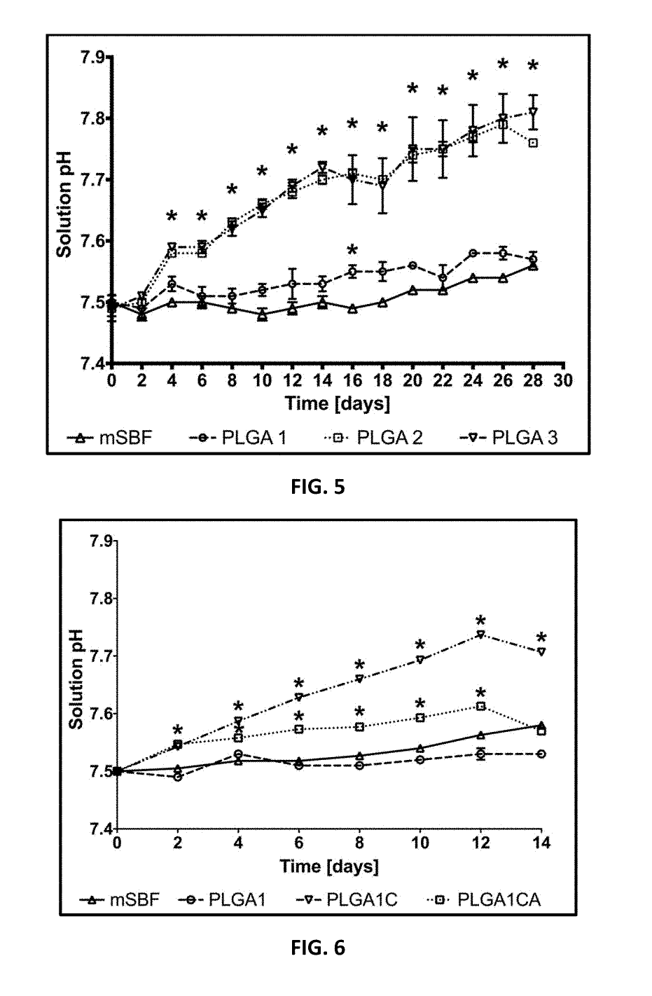

In order to determine the effect of subchondral bone implant degradation on local pH, the pH of the mSBF bathing solution of the implants was measured every two days throughout the duration of the study. For non-compacted implants (FIG. 5), the pH of mSBF from both PLGA2 and PLGA3 demonstrated significant increases when compared to corresponding mSBF controls at all time points evaluated from Day 4 through Day 28. Conversely, the pH of mSBF containing PLGA1 constructs did not demonstrate significant change in pH compared to mSBF controls at any time point, with the exception of a marginally significant increase at Day 16 (p=0.0455).

For compacted implants (FIG. 6), the pH of mSBF containing PLGA1C implants significantly increased when compared to corresponding mSBF controls at all time points evaluated from Day 2 to Day 14. The pH range of mSBF bathing the PLGA1C implants (7.50-7.74) was higher than that of PLGA1 (7.50-7.58).

The pH of mSBF containing compacted and annealed subchondral bone implants (PLGA1CA) significantly increased compared to the mSBF controls at all time points evaluated from Day 2 to Day 12, but was not significantly different by Day 14. However, the pH range of mSBF bathing PLGA1CA implants (7.50-7.61) was lower than PLGA1C (7.50-7.74), respectively (FIG. 6).

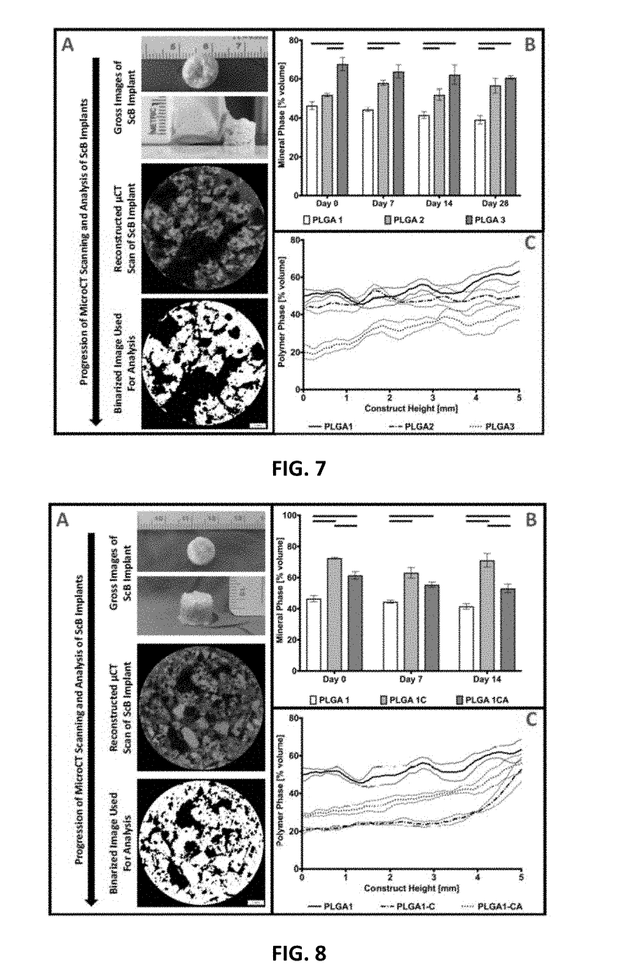

MicroCT analysis of implants prior to and following degradation in mSBF was used to quantify mineral phase volume. At FIG. 7 and FIG. 8, section A is provided representative images of the progressive stages of micro CT analysis including top and side images of a representative implant, reconstructed micro CT scan of an implant, and a binarized images of a micro CT scan used for analysis. Variations in mineral phase between the different non-compacted implants is shown in FIG. 7 at B. Comparing across the implants, PLGA1 mineral phase volume (46.5±2.05%) was lower compared to PLGA2 (52.0±0.85%, p=0.3286) and significantly lower compared to PLGA3 (67.7±3.39%, p<0.0001) at Day 0. At Day 28, PLGA1 mineral phase volume (39.2±2.17%) was significantly lower than both PLGA2 (56.6±3.65%, p=0.0003) and PLGA3 (60.6±0.86%, p<0.0001). The polymer phase distribution was fairly heterogenous throughout the height of all three implant formulations (FIG. 7, C).

For the compacted implants (PLGA1C), mineral phase volume (72.5±0.44%) was significantly higher compared to PLGA1 (46.5±2.05%) at Day 0 (p<0.0001) (FIG. 8 at B) at Day 0. At Day 14, mineral phase volume for PLGA1C (71.14±4.35%) was significantly higher compared to PLGA1 (41.6±1.74%) (p<0.0001). In addition to the increase in mineral phase volume for compacted ScB implants, the polymer phase distribution was more homogeneous throughout the height of the implant compared to PLGA1 (FIG. 8 at C).

For the compacted and annealed implants (PLGA1CA), mineral phase volume (61.5±2.36%) was significantly lower compared to PLGA1C (72.5±0.44%) (p=0.0143) (FIG. 8 at B) at Day 0. At Day 14, mineral phase volume for PLGA1CA (41.6±2.80%) was significantly lower compared to PLGA1C (71.1±4.35%) (p=0.0002). Despite the decrease in mineral phase volume for compacted and annealed ScB implants, the polymer phase distribution was comparable in homogeneity throughout the height of the implant compared to PLGA1C (FIG. 8 at C).

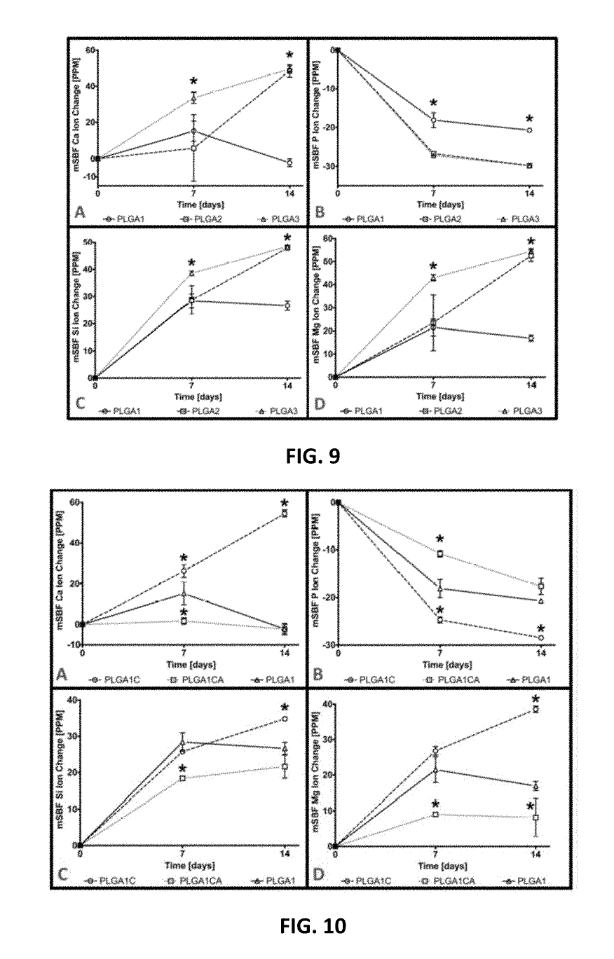

Changes in calcium, phosphorous, silica, and magnesium ion concentrations within mSBF solutions bathing the subchondral bone implants were evaluated using ICP-AES to assess implant bioactivity. Bioactivity was indicated by a decrease in solution ion concentration of phosphorous and an increase in solution ion concentrations calcium, silica, and magnesium. Calcium ion concentration of mSBF without implants at Day 0 was 101.91±0.71 ppm. At Day 7, the calcium ion concentration of mSBF bathing PLGA1, PLGA2, and PLGA3 implants increased by 15.21±5.52 ppm, 5.84±18.36 ppm, and 33.50±2.97 ppm, respectively. All three changes were significant compared to the Day 0 mSBF control, with PLGA3 bathing solution exhibiting the greatest increase in calcium ion concentration. At Day 14, the calcium ion concentration of mSBF bathing PLGA1, PLGA2, and PLGA3 decreased by 2.23±2.18 ppm, increased by 48.51±3.54 ppm, and increased 49.59±1.64 ppm, respectively. PLGA2 and PLGA3 bathing solutions exhibited a significant change from the Day 0 mSBF control but were not significantly different from each other (FIG. 9 at A). Phosphorous ion concentration of mSBF without implants at Day 0 was 33.42±0.21 ppm. At Day 7, the phosphorous ion concentration of mSBF bathing PLGA1, PLGA2, and PLGA3 implants decreased by 18.12±1.92 ppm, 26.69±0.28 ppm, and 27.15±0.18 ppm, respectively. All three changes were significant compared to the Day 0 mSBF control, with PLGA2 and PLGA3 having significantly greater change compared to PLGA1. At Day 14, the phosphorous ion concentration of mSBF bathing PLGA1, PLGA2, and PLGA3 implants decreased by 20.71±0.28 ppm, 29.79±0.19 ppm, and 29.82±0.21 ppm respectively. All three changes were significant compared to the Day 0 mSBF control, with PLGA2 and PLGA3 having significantly greater change compared to PLGA1 (FIG. 9 at B). Silica ion concentration of mSBF without implants at Day 0 was 1.74±0.03 ppm. At Day 7, the silica ion concentration of mSBF bathing PLGA1, PLGA2, and PLGA3 implants increased by 28.39±2.55 ppm, 28.64±5.30 ppm, and 38.57±0.84 ppm, respectively. All three changes were significant compared to the Day 0 mSBF control, with PLGA3 having significantly greater change compared to PLGA1 and PLGA2. At Day 14, the silica ion concentration of mSBF bathing PLGA1, PLGA2, and PLGA3 implants increased by 26.62±1.68 ppm, 47.91±0.67 ppm, and 48.49±0.23 ppm, respectively. All three changes were significant compared to the Day 0 mSBF control, with PLGA2 and PLGA3 having significantly greater change compared to PLGA1 (FIG. 9 at C). Magnesium ion concentration of mSBF without implants at Day 0 was 45.50±0.33 ppm. At Day 7, magnesium ion concentration of mSBF bathing PLGA1, PLGA2, and PLGA3 implants increased by 21.48±3.63 ppm, 23.48±12.05 ppm, and 42.98±1.35 ppm, respectively. All three changes were significant compared to the Day 0 mSBF control, with PLGA3 having significantly greater change compared to PLGA1 and PLGA2. At Day 14, magnesium ion concentration of mSBF bathing PLGA1, PLGA2, and PLGA3 implants increased by 16.89±1.30 ppm, 52.57±2.45 ppm, and 54.47±1.15 ppm respectively. All three changes were significant compared to the Day 0 mSBF control, with PLGA2 and PLGA3 having significantly greater change compared to PLGA1 (FIG. 9 at D).

For the compacted implant (PLGA1C) degradation, the calcium ion concentration of mSBF without implants at Day 0 was 104.40±0.39 ppm. Calcium ion concentration of mSBF bathing PLGA1C increased by 26.31±3.16 ppm and 54.44±1.55 ppm at Day 7 and Day 14, respectively (FIG. 10 at A). Phosphorous ion concentration of mSBF without implants at Day 0 was 31.68±0.02 ppm. Phosphorous concentration of mSBF bathing PLGA1C decreased by 24.72±0.63 ppm and 28.48±0.15 ppm at Day 7 and Day 14, respectively (FIG. 10 at B). Silica ion concentration of mSBF without implants at Day 0 was 12.35±0.06 ppm. Silica ion concentration of mSBF bathing PLGA1C increased by 25.74±0.46 ppm and 34.80±0.31 ppm at Day 7 and Day 14, respectively (FIG. 10 at C). Magnesium ion concentration of mSBF without implants at Day 0 was 38.39±0.18 ppm. Magnesium ion concentration of mSBF bathing PLGA1C increased by 26.80±1.22 and 38.56±0.90 at Day 7 and Day 14, respectively (FIG. 10 at D).

For the compacted and annealed (PLGA1CA) implant degradation, the calcium ion concentration of the mSBF without implants at Day 0 was 104.40±0.39 ppm. The calcium ion concentration of mSBF bathing PLGA1CA increased by 1.71±1.58 ppm at Day 7 and decreased by 2.28±2.85 ppm at Day 14. (FIG. 10 at A). Phosphorous ion concentration of mSBF without implants at Day 0 was 31.68±0.02 ppm. Phosphorous concentrations of mSBF bathing PLGA1CA decreased by 10.82±0.66 ppm and 17.66±1.72 ppm at Day 7 and Day 14 respectively. (FIG. 10 at B). Silica ion concentration of mSBF without implants at Day 0 was 12.35±0.06 ppm. Silica ion concentration of mSBF bathing PLGA1CA increased by 18.36±0.58 ppm and 21.62±3.19 ppm at Day 7 and Day 14 respectively (FIG. 10 at C). Magnesium ion concentration of mSBF without implants at Day 0 was 38.39±0.18 ppm. Magnesium ion concentration of mSBF bathing PLGA1CA was increased by 9.03±0.40 ppm and 8.15±5.26 ppm at Day 7 and Day 14, respectively (FIG. 10 at D).

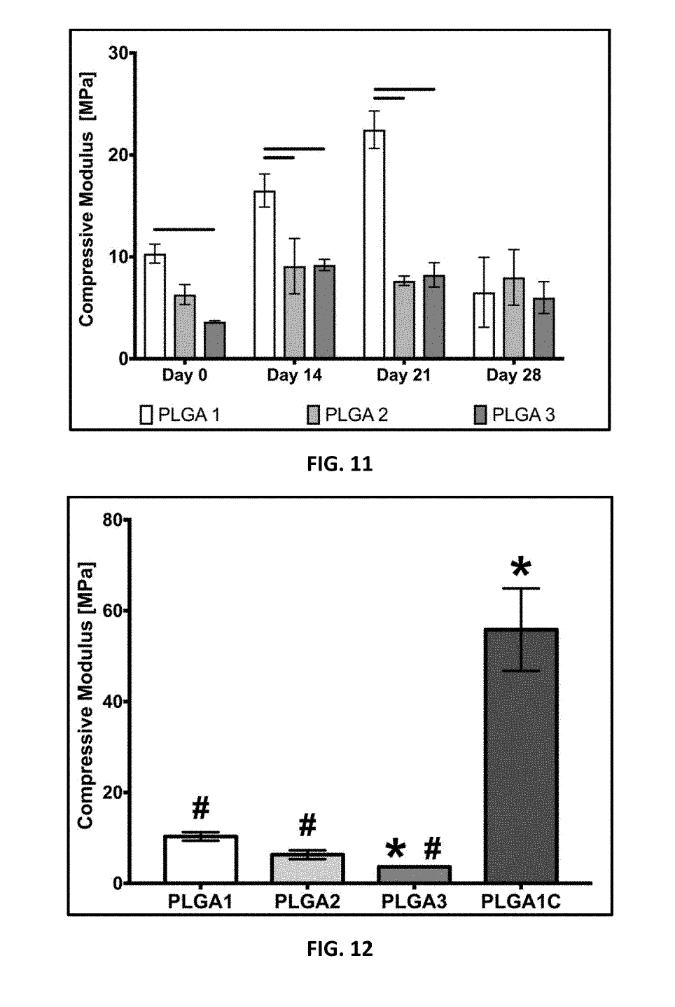

Compressive modulus was evaluated via unconfined compression testing in a saline bath. For the Phase I degradation study (FIG. 11), compressive modulus was not significantly different for PLGA1 (10.31±0.93 MPa) compared to PLGA2 (6.30±0.98 MPa, p=0.2745) but was significantly greater compared to PLGA3 (3.63±0.11 MPa, p=0.0377) at Day 0. PLGA1 compressive modulus (6.52±3.43 MPa) was not significantly different compared to PLGA2 (7.99±2.72 MPa, p=0.8345) or PLGA3 (6.01±1.56 MPa, p=0.9774) at Day 28.

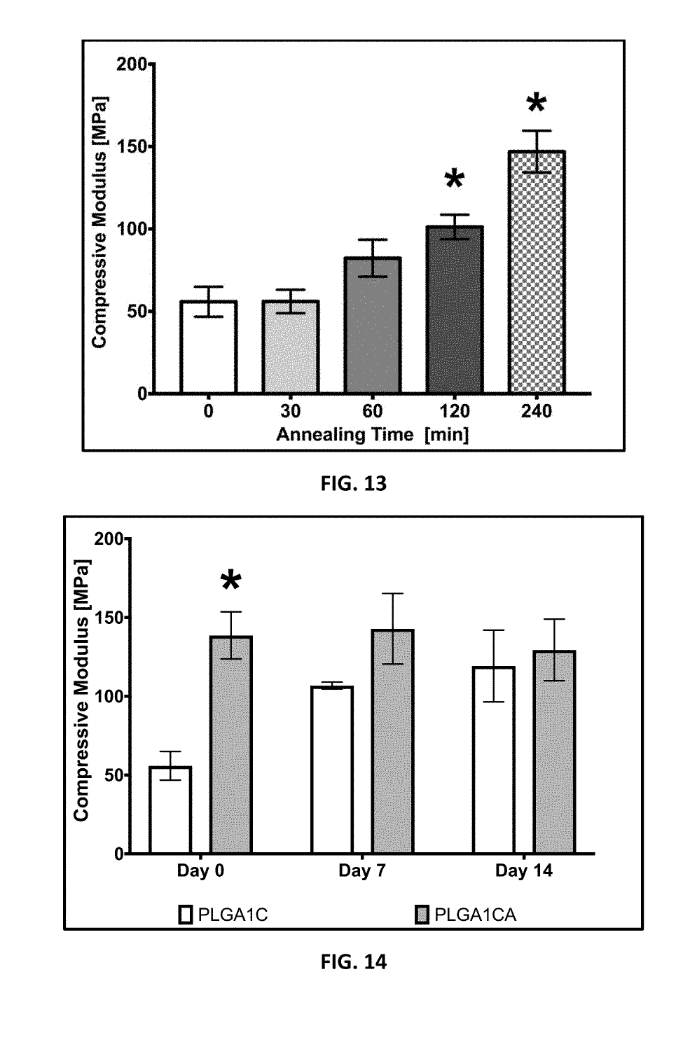

Compressive modulus for PLGA1C implants (55.83±9.09 MPa) was significantly greater than that of the original PLGA1 formulation (10.31±0.93 MPa, p=0.0007) (FIG. 12). Compressive modulus was not significantly different for PLGA1C implants that were annealed for 30 minutes (56.01±7.09 MPa, p=0.9897) or 60 minutes (82.27±11.22 MPa, p=0.0691). However, compressive modulus was significantly higher for implants that were annealed for 120 minutes (101.21±7.43 MPa, p=0.0036) and 240 minutes (146.92±12.63 MPa, p<0.0001) (FIG. 13). When compressed, implants that were not compacted or annealed flattened indefinitely. While compacted and annealed implants exhibited brittle fracture behavior.

For the second degradation study (FIG. 14), compressive modulus was significantly greater for PLGA1CA (138.68±14.93 MPa) than PLGA1C (55.83±9.09 MPa, p=0.0139) at Day 0. Compressive modulus trended higher for PLGA1CA (129.42±19.53 MPa) than PLGA1C (119.20±22.75 MPa, p=0.9662) at Day 28, though significant differences were not seen.

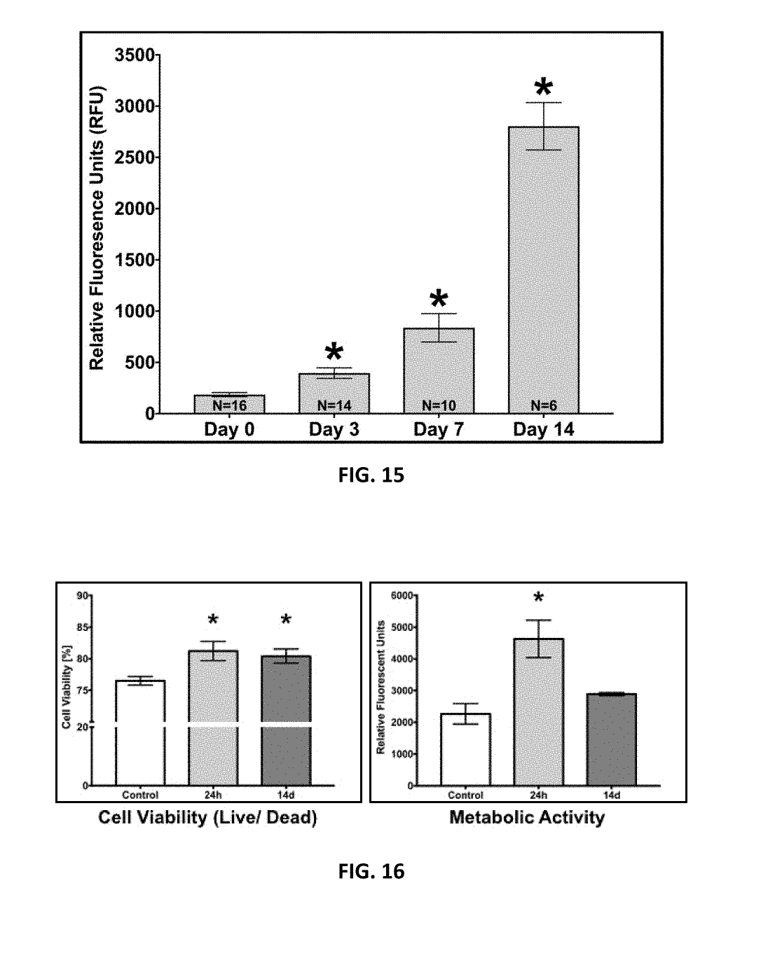

The AB assay and live/dead imaging indicated a low initial seeding density on the ScB implants. Cell viability and proliferation increased significantly throughout the 14-day study, suggesting that the material is cytocompatible. Compared to fluorescence at Day 0 (184.90±19.72 relative fluorescent units (RFU)), a significant increase in fluorescence was seen in Day 3 implants (395.47±51.60 RFU, p<0.0001), Day 7 implants (837.82±136.36 RFU, p<0.0001) and Day 14 implants (2803.67±230.91 RFU, p<0.0001), indicating cell viability (FIG. 15). Live/dead fluorescent imaging also depicted a significant increase in cellular proliferation over the 14-day study as well as 3D infiltration of the cells.

### EXAMPLE 2

Osteochondral plugs (OCPs) were made with three distinct layers: ScB, calcified cartilage, and cartilage. For the ScB layer, PLGA1C implants were made as described above in Example 1.

For the cartilage layer, a cartilage analog (CA) was made as described in U.S. Patent Publication No. 2018,0256784 to Fernandez, et al., previously incorporated herein by reference. Briefly, nucleus pulposus tissue was removed from the center of the intervertebral discs of bovine tails. Decellularization solution was made in 50 mM Tris buffer (pH 7.5) containing 2% antibiotic/antimycotic. The decellularization solution included 1.2% Triton X-100, 0.2% EDTA, 0.02% sodium azide, and 50 mM Tris all in 400 mL DI water with a pH of 7.5. The tissue was placed in the decellularization solution and subjected to ultrasonication for 10 minutes prior to undergoing constant agitation (150 RPM) on an orbital shaker at room temperature. Every twenty-four hours thereafter the solution was changed and the tissue was again subjected to ultrasonication for an additional 10 minutes. This process was repeated for a total of either three days. At the completion of the process, the tissue was rinsed in sequential changes of distilled water, 70% ethanol and distilled water again; each for 30 minutes under orbital agitation (150 RPM) at room temperature prior to undergoing nuclease treatment (720 mU/ml DNase and RNase) at 37° C. for 48 hours. All nuclease solutions were made in 1× PBS (pH 7.5) containing 5 mM magnesium chloride. Following nuclease treatment the tissue was thoroughly rinsed in distilled water for one hour under agitation.

Following formation, CAs were frozen at −80° C., lyophilized for 48 hours at 0.100 mBar and -84° C., and stored under desiccation. For the calcified cartilage layer (CCL), 2.5 g of PLGA was dissolved in 5 mL acetone followed by mixing with 5 g of hydroxyapatite powder (diameter<100 μm). Approximately 0.1 mL of CCL was spread on top of the ScB implant. A lyophilized CA was then compacted onto the CCL for 15 seconds with 2.5 lbs of compressive force. The ScB/CCL/CA implants were dried for 48 hrs at RT before annealing in an oven for 240min at 115° C. OCPs were stored under desiccation until needed. OCPs were sterilized with ethylene oxide (EO) in an Anprolene AN74i tabletop sterilizer for 12 hours, followed by a 48-hour degassing period.

OCP cytocompatibility was assessed indirectly with guidance from ISO 10993. Conditioned media was created by soaking sterilized OCPs in standard CCM (10% (v/v) FBS, 1% (v/v) Ab/Am, 1% (v/v) GLUT, and DMEM) at 37° C. for either 24 hours or 14 days (n=3/time point; 1 mL CCM per 3 cm² of OCP surface area per ISO 10993-12). Passage 6 (P6) hMSC-BMs were seeded at a density of 1×10⁴ cells/well in a 96-well plate with 100 μL of CCM and incubated for 24 hours at 37° C. with 5% CO₂ to ensure attachment. Following incubation, the media was aspirated, replaced with 100 μm of conditioned media, and cells were returned to incubate for 24 hours. Cells cultured in a 1% (v/v) solution of Triton X-100 in parallel with experimental groups was used as a positive death control. Cell metabolic activity was assessed with an AB assay. Cells were incubated for 2 hours in CCM supplemented with 10% (v/v) AB fluorescent dye. Fluorescence of AB was measured at an excitation wavelength of 545 nm and an emission wavelength of 590 nm using a fluorescence microplate reader. CCM supplemented AB dye was used as a negative control and its fluorescent intensity subtracted from experimental values. Cell viability was further assessed with a live/dead fluorescent assay. After removing AB supplemented CCM, cells were rinsed 3× with sterile PBS then submerged in a solution of 2 μM calcein acetotoxymethyl, 4 μM ethidium homodimer-1, and 1 μL 4,6-Diamidino-2-phenylindole (DAPI) in PBS for 45 minutes at room temperature. Samples were rinsed and submerged in PBS and imaged with a fluorescent microscope.

The control group fluorescence was 2267±322.8 relative fluorescent units (RFU). Implants degraded for 24 h had a significant increase in fluorescence (4629±584.9RFU, p=0.0089) while implants degraded for 14 d were not significantly different (2889±46.7RFU, p=0.4624) compared to controls. A live/dead fluorescent assay (FIG. 15) was also used to semi-quantitatively assess cell viability. FIG. 16 presents comparison of hMSC-BM cell viability determined using a live/dead fluorescent assay (left) and comparison of cellular metabolic activity determined using an AB assay (Right). (Star (*) indicates significant difference (p<0.05).) Control groups had a viability of 76.51±0.39%, which was significantly lower than that of the 24 h group (81.22±0.88%, p=0.0045) and the 14 d group (80.41±0.65, p=0.0110).

### EXAMPLE 3

CA materials formed as described above in Example 2 were compressed and crosslinked using a hyperosmotic pressure gradient as schematically illustrated in FIG. 2. First, 1200 mL of hyperosmotic solution was made in distilled water containing 50 mM 2-(N-morpholino)ethanesulfonic acid (MES) buffer and 114.87 g of polyethylene glycol (PEG) (20 kDa molecular weight) and adjusted to a pH of 5.5. Based on the biochemical composition of the CA, this solution was estimated to produce approximately 0.01 MPa of compressive osmotic pressure on the CA when submerged.

CAs were individually placed into dialysis tubing (12-14 kDa molecular weight cutoff, Spectrum Labs, Rancho Dominguez, Calif.) and sealed with a clamp at each end. For each batch, six sealed dialysis tubes containing CAs were placed in a beaker along with 1200 mL of hyperosmotic solution and placed on an orbital shake plate for 24 h at 150 rpm to compact the CA implants. Dialysis tubing with compacted CAs were subsequently placed in a beaker along with 1200 mL of crosslinking solution made with distilled water containing 50 mM MES buffer, 114.87 g PEG, 30 mM 1-ethyl-3-(3-dimethylaminopropyl)carbodiimide (EDC), and 6 mM N-hydroxysuccinimide (NHS) and adjusted to a pH of 5.5. The beaker was again placed on an orbital shake plate for 24 h at 150 rpm. After the crosslinking step, hyperosmotically crosslinked cartilage analogs (HOX-CAs) were removed from dialysis tubing and rinsed in 100 mL of distilled water for 1 h, 100 mL of 70% ethanol for 30min, and 100 mL of distilled water for 30 min, all on an orbital shake plate at 150 rpm. All HOX-CA samples were stored in a storage solution (1× PBS, 2% (v/v) Ab/Am, 1× protease inhibitor, and 0.02% (w/v) sodium azide) until needed for further studies.

HOX-CA samples were placed in a 24-well tissue culture plate. Samples were frozen at −80° C. for 24 h. Samples were subsequently placed in a chamber lyophilizer (−87° C. and 0.100 atm) for 48 hours. After lyophilization, samples were stored under desiccation until needed.

HOX-OCPs were then formed using PLGA1C materials as described in Example 1 and lyophilized HOX-CA materials. HOX-OCPs were fabricated with three distinct layers: PLGA1C, CCL, and HOX-CA. Approximately 0.1 mL of CCL was spread on top of the PLGA1C implant and a lyophilized HOX-CA was compacted onto the CCL for 15 seconds with 2.5 lbs. The PLGA1C/CCL/HOX-CA implants were dried for 48 hrs at room temperature and designated HOX-OCP. Following fabrication, samples were annealed in an oven at 115° C. for 4 h, and designated as HOX-OCP-A.

DMA was performed to characterize the viscoelastic properties (complex, storage, and loss moduli, phase angle, and hysteresis) of HOX-CA (n=5), HOX-OCP (n=6), and HOX-OCP-A (n=5) materials. Testing was performed on a TA ElectroForce 3200 Series II electromechanical test frame running WinTest7 software with a DMA Analysis package. Prior to testing, materials were hydrated for 48 hours in 1× phosphate buffered saline (PBS). OCPs were inserted between two stainless steel platens in a PBS bath, preloaded with 10 g of compressive load to ensure specimen contact, and allowed to equilibrate for 1200 s under constant displacement. Physiological load magnitudes induce different strains at different frequencies. Thus, a physiologically relevant strain-based loading regime was applied to all samples, with compressive strain applied as follows: 10 cycles of 0%-16% strain at 0.1 Hz, 10 cycles of 0%-13% strain at 1 Hz, 10 cycles 0%-10% strain at 10 Hz, and 40 cycles of 0%-9% strain at 40 Hz, with a 1200 s relaxation time between each frequency.

Interfacial strength testing was performed with a custom shear test fixture fitted to a TA Electroforce 3200 Series II electromechanical test frame. HOX-OCP and HOX-OCP-A implants were hydrated in 1× PBS for 48 hours prior to testing. Implants were loaded into a confining chamber until the cartilage/calcified cartilage interface was exposed. A containment plug was inserted flush with OCP base to prevent implant migration during testing. Samples were tested to failure under compression using a custom shear crosshead and 1000 g load cell at a strain rate of 10% min⁻¹. Shear stiffness was calculated using the slope of the linear region of the force/displacement curve before failure.

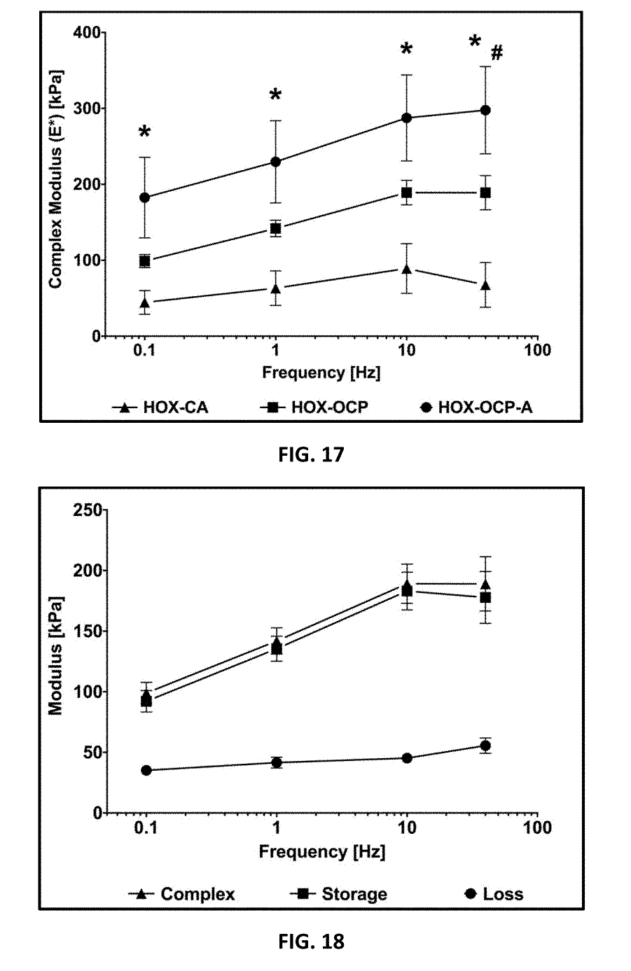

Attachment of the HOX-CA to the ScB implant (HOX-OCP) and subsequent annealing (HOX-OCP-A) both increased the complex and storage moduli compared to unattached HOX-CA, though statistically significant increases only occurred at certain frequencies (Table 2, FIG. 17). (On FIG. 17, star (*) indicates a significant difference from HOX-CA implants (p<0.05). Pound (#) indicates a significant difference from HOX-OCP implants (p<0.05).)

TABLE 2

| | | Dynamic Modulus | | | | |
|---|---|---|---|---|---|---|
| Frequency | | Complex | Storage | Loss | Phase Angle | Hysteresis |
| [Hz] | Sample | [kPa] | [kPa] | [kPa] | [degrees] | [g-mm] |
| 0.1 | HOX-CA | 44.53 ± 15.70 | 39.36 ± 12.98 | 20.02 ± 9.27 | 24.04 ± 2.34 | 4.65 ± 1.74 |
| | HOX-OCP | 99.01 ± 8.73 | 92.10 ± 8.85 | 35.11 ± 3.90 | 21.23 ± 2.34 | 7.37 ± 0.89 |
| | HOX-OCP-A | 182.60 ± 53.03* | 179.35 ± 53.11* | 32.00 ± 4.81 | 12.42 ± 1.97* | 5.86 ± 0.89 |
| 1 | HOX-CA | 63.32 ± 22.88 | 59.18 ± 21.18 | 21.92 ± 8.96 | 19.34 ± 2.42 | 3.01 ± 0.99 |
| | HOX-OCP | 141.89 ± 10.86 | 135.44 ± 10.38 | 41.51 ± 4.56 | 17.06 ± 1.30 | 5.98 ± 1.13* |
| | HOX-OCP-A | 229.64 ± 54.12* | 223.62 ± 54.95* | 46.73 ± 4.24 | 14.59 ± 2.65 | 5.46 ± 0.22 |
| 10 | HOX-CA | 89.15 ± 32.71 | 83.98 ± 30.73 | 28.48 ± 11.16 | 18.31 ± 1.86 | 2.26 ± 0.71 |
| | HOX-OCP | 189.17 ± 16.15 | 183.12 ± 15.60 | 45.21 ± 4.28 | 13.79 ± 0.45 | 3.76 ± 0.49 |
| | HOX-OCP-A | 287.46 ± 56.63* | 281.98 ± 57.77* | 50.79 ± 3.41 | 12.31 ± 2.55 | 3.46 ± 0.17 |
| 40 | HOX-CA | 67.65 ± 29.47 | 54.04 ± 26.48 | 36.57 ± 12.93 | 39.30 ± 6.16 | 2.55 ± 0.68 |
| | HOX-OCP | 189.01 ± 22.42* | 177.80 ± 21.36* | 55.54 ± 6.33 | 17.21 ± 0.80* | 4.45 ± 0.59 |
| | HOX-OCP-A | 297.67 ± 57.44*# | 283.98 ± 57.77*# | 72.82 ± 6.40* | 15.87 ± 2.28* | 4.41 ± 0.35 |

Star (*) indicates a significant difference from HOX-CA implants (p < 0.05).
Pound (#) indicates a significant difference from HOX-OCP implants (p < 0.05).

Complex and storage moduli of each implant group were greater in magnitude compared to the respective loss modulus (FIG. 18). HOX-OCP implants had lower phase angles compared to HOX-CA implants, with a significantly lower phase angle at 40 Hz (17.21°±0.80° versus 39.30°±6.16°, p<0.0001). HOX-OCP-A implants had lower phase angles compared to HOX-CA implants, with significantly lower phase angles at 0.1 Hz (12.24±1.97° versus 24.04°±2.34°, p=0.0164) and 40 Hz (15.87°±2.28° versus 39.30°±6.16°, p<0.0001). HOX-OCP implants had greater hysteresis values compared to HOX-CA implants, though only significantly at 1 Hz (5.98±1.13 g-mm versus 3.01±0.99 g-mm, p=0.0400). HOX-OCP-A implants trended to have greater hysteresis values compared to HOX-CA implants, though not statistically different.

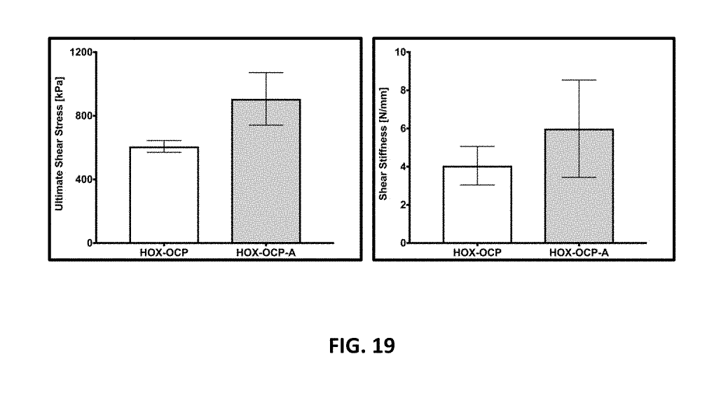

HOX-OCP-A implants (906.36±165.02 kPa) had greater, though not significantly different, interfacial shear strength compared to HOX-OCP implants (607.02±36.75 kPa) (p=0.1978) (FIG. 19); (Left) Ultimate shear stress of HOX-OCP and HOX-OCP-A implants. (Right) Shear stiffness of HOX-OCP and HOX-OCP-A implants.

While certain embodiments of the disclosed subject matter have been described using specific terms, such description is for illustrative purposes only, and it is to be understood that changes and variations may be made without departing from the spirit or scope of the subject matter.

What is claimed is:

1. A synthetic biocompatible subchondral bone analog material comprising a polymeric matrix, first particles comprising a calcium phosphate held in the polymeric matrix, and second particles comprising a bioactive glass held in the polymeric matrix.

2. The synthetic biocompatible subchondral bone analog material of claim 1, the polymeric matrix comprising a biodegradable polymer.

3. The synthetic biocompatible subchondral bone analog material of claim 2, the biodegradable polymer comprising a poly(lactic acid), a poly(glycolic acid), a poly(lactic-co-glycolic acid), a poly(ortho ester), a polyanhydride, a polycaprolactone, a polysulfone, a polyolefin, a polyvinyl alcohol, a polyalkenoic, a polyacrylic acid, a polyester, or a combination of two or more polymers.

4. The synthetic biocompatible subchondral bone analog material of claim 1, comprising the polymeric matrix in an amount of from about 15 wt. % to about 50 wt. % of the material.

5. The synthetic biocompatible subchondral bone analog material of claim 1, the calcium phosphate comprising a hydroxy apatite.

6. The synthetic biocompatible subchondral bone analog material of claim 1, wherein about 90 wt. % or more of the first particles are in a size range of from about 1000 μm to about 2000 μm.

7. The synthetic biocompatible subchondral bone analog material of claim 1, comprising the first particles in an amount of from about 25 wt. % to about 50 wt. % of the material.

8. The synthetic biocompatible subchondral bone analog material of claim 1, wherein about 90 wt. % or more of the second particles are in a size range of from about 20 μm to about 200 μm.

9. The synthetic biocompatible subchondral bone analog material of claim 1, comprising the second particles in an amount of from about 20 wt. % to about 40 wt. % of the material.

10. The synthetic biocompatible subchondral bone analog material of claim 1, wherein the material is compacted and/or annealed.

11. An osteochondral implant comprising the synthetic biocompatible subchondral bone analog material of claim 1.

12. A multilayered osteochondral implant comprising:
a first layer comprising a synthetic biocompatible subchondral bone analog material that includes a first polymeric matrix, first particles comprising a first calcium phosphate in the polymeric matrix, and second particles comprising a bioactive glass in the polymeric matrix;
a second layer comprising a calcified cartilage analog material that includes a second polymeric matrix and third particles comprising a second calcium phosphate in the second polymeric matrix; and
a third layer comprising a cartilage analog material.

13. The multilayered osteochondral implant of claim 12, wherein the first polymeric matrix and the second polymeric matrix both comprise a homopolymer or a copolymer of poly(lactic acid) and/or poly(glycolic acid).

14. The multilayered osteochondral implant of claim 12, wherein the first calcium phosphate and the second calcium phosphate both comprise a hydroxy apatite.

15. The multilayered osteochondral implant of claim 12, wherein about 90 wt. % or more of the first particles are in a size range of from about 1000 μm to about 2000 μm and/or wherein about 90 wt. % or more of the third particles are about 100 μm or less in size.

16. The multilayered osteochondral implant of claim 12, the second layer comprising from about 50 wt. % to about 90 wt. % of the third particles.

17. The multilayered osteochondral implant of claim 12, the cartilage analog material of the third layer comprising a decellularized natural tissue.

18. The multilayered osteochondral implant of claim 12, wherein the cartilage analog material is compressed and/or crosslinked.

19. The multilayered osteochondral implant of claim 18, wherein the cartilage analog has been compressed and/or crosslinked according to a hyperosmotic process.

20. The multilayered osteochondral implant of claim 12, wherein the implant is annealed.
